(19) United States
(12) Patent Application Publication
(10) Pub. No.: US 2023/0272817 A1
(43) Pub. Date: Aug. 31, 2023

(54) SEGMENTED ADJUSTBLE ARM WITH LOCKING MECHANISM

(71) Applicant: Noga Engineering & Technology (2008) Ltd., Shlomi (IL)

(72) Inventors: Eitan YEHUD, Nahariya (IL); Shay Raviv, Kiryat Motzkin (IL); Michel Allouch, Yoqneam IIit (IL)

(73) Assignee: Noga Engineering & Technology (2008) Ltd., Shlomi (IL)

(21) Appl. No.: 17/683,316

(22) Filed: Feb. 28, 2022

(51) Int. Cl. F16C 11/10; F16M 11/14

(52) U.S. Cl. CPC F16C 11/106; F16M 11/14; A61B 90/50

(57) ABSTRACT

A segmented adjustable arm device comprising a segment chain of a plurality of interconnected segments, wherein adjacent ends of each pair of adjacent segments of the segment chain form a ball and socket joint, one of the adjacent ends of each pair of the adjacent segments presenting a spherical or partially spherical plug and the other end of the adjacent ends presenting a socket configured to receive and hold the plug, allowing relative radial motion between the segments of that pair; and a locking mechanism, including a locking train that includes one or a plurality of rigid elements arranged in series along the segment chain, configured, when subjected to applied pressure, to pull the plurality of interconnected segments away from each other, and increase friction at the ball and socket joint to immobilize motion between adjacent segments.

### FIELD OF THE INVENTION

The present invention relates to adjustable arms. More particularly, the present invention relates to a segmented adjustable arm with a locking mechanism.

### BACKGROUND OF THE INVENTION

Adjustable arms are typically provided in a form of an assembly of interconnected parts, that form an elongated body that may be shaped and reshaped in a variety of alignments, and may be immobilized in a desired alignment. Adjustable arms may have various uses. For example, an adjustable arm may be used to hold a load, such as a screen (e.g., TV set), a tool (e.g., a dental drill) at a particular position in space, by aligning the adjustable arm in a desired alignment and fixing it in that alignment. Adjustable arms may also be used to point at or direct to a certain target in space, for example, by holding at a distal end of the adjustable arm the nozzle of a hose, directing a fluid flow emerging from the nozzle to a particular target. In some examples, the adjustable arm may include an internal conduit through which fluid is allowed to flow and be dispensed through a nozzle at a distal end of the adjustable arm.

An adjustable arm may include a plurality of interconnected jointed segments so that pairs of adjacent segments may be tilted with respect to one another to assume a desirable angle and be fixed in that position.

Adjustable arms may be useful in applications where a user wants to temporarily fix the arm in a desired alignment, and later change the alignment of the adjustable arm for another purpose. For example, a photographer may attach a camera to an adjustable arm, aligning the adjustable arm to face a desirable angle of view, and later on reshape the adjustable arm and fix it in another alignment so as to direct the camera in another angle of view. In another example, a physician may find an adjustable arm useful when performing a medical procedure, where the adjustable arm may be used as an assisting tool in holding one or more medical tools.

Adjustable arms may also be used in machining to direct a flow of high-pressure coolant, onto a workpiece being machined. Applying high-pressure coolant on the workpiece may lubricate the workpiece effectively and cool the workpiece efficiently as the machining tool processes the workpiece. Additionally, high-pressure coolant may be used to increase heat dissipation and to expel metal shards and shavings (byproducts of machining) from the workpiece and the machining tool.

Sometimes, when high-pressure fluid (e.g., coolant) is transferred via an adjustable arm, the arm may lose its initial user-inflicted curvature and straighten up due to the enormous pressure of fluid within the adjustable arm.

It may be desirable to provide a segmented adjustable arm that can be manipulated to assume a desired shape and locked in that shape, so as to withstand considerably large forces (e.g., heavy load, flow of high-pressure coolant through the adjustable arm, etc.) and may be locked in position or released in a simple manner.

### SUMMARY OF THE INVENTION

There is thus provided, in accordance with an embodiment of the invention, a segmented adjustable arm device includes a segment chain comprising a plurality of interconnected segments, wherein adjacent ends of each pair of adjacent segments of the segment chain form a ball and socket joint, one of the adjacent ends of each pair of the adjacent segments presenting a spherical or partially spherical plug and the other end of the adjacent ends presenting a socket configured to receive and hold the plug, allowing relative radial motion between the segments of that pair and a locking mechanism that includes a locking train that includes one or a plurality of rigid elements arranged in series extending along the segment chain, configured, when subjected to applied pressure, to pull the plurality of interconnected segments away from each other, and increase friction at the ball and socket joint between each pair of the adjacent segments so as to immobilize motion between adjacent segments of the segment chain to fix the segmented adjustable arm in a desired alignment, and configured, when the pressure is released, to reduce the friction at the ball and socket joint between each pair of the adjacent segments of the segment chain, to render the segmented adjustable arm pliable; and a pressure applicator to apply or release the pressure.

According to some embodiments of the invention, the locking train is configured, when subjected to applied pressure, to push a distal segment of the segment chain.

According to some embodiments of the invention, the locking train is inserted within the segment chain.

According to some embodiments of the invention, the locking train extends within a lumen defined within the segment chain.

According to some embodiments of the invention, the plurality of rigid elements comprises balls arranged in series.

According to some embodiments of the invention, the plurality of rigid elements comprises balls and tubular members arranged in series.

According to some embodiments of the invention, a bore extends within the locking train, defining an internal passage.

According to some embodiments of the invention, a tube extending within the internal passage.

According to some embodiments of the invention, the tube comprises an inlet at a proximal portion of the adjustable arm, and an outlet at a distal portion of the adjustable arm.

According to some embodiments of the invention, the outlet is located at a distal end of the adjustable arm.

According to some embodiments of the invention, the pressure applicator comprises a plunger for applying the pressure on one of the rigid elements of the locking train.

According to some embodiments of the invention, the pressure applicator comprises a screw configured to actuate the plunger.

According to some embodiments of the invention, the screw is oriented substantially orthogonally to the plunger.

According to some embodiments of the invention, the plunger is hollow, forming part of the internal passage.

According to some embodiments of the invention, the device further includes a second locking train.

According to some embodiments of the invention, the second locking train is configured to operate in a direction opposite to a direction of operation of the locking train.

### BRIEF DESCRIPTION OF THE DRAWINGS

In order for the present invention to be better understood and for its practical applications to be appreciated, the following Figures are provided and referenced hereafter. It should be noted that the Figures are given as examples only and in no way limit the scope of the invention. Like components are denoted by like reference numerals.

FIG. 1 is a side view of a segmented adjustable arm with a locking mechanism, in accordance with some embodiments of the present invention.

FIG. 2 is cross section view of the segmented adjustable arm with locking mechanism of FIG. 1.

FIG. 3 is an isometric view of a segmented adjustable arm with a locking mechanism, with an internal conduit for delivering and dispensing fluid, in accordance with some embodiments of the present invention.

FIG. 4 is a cross sectional view of the segmented adjustable arm with locking mechanism of FIG. 3.

FIG. 5 is an exploded view of the segmented adjustable arm with locking mechanism of FIG. 3.

FIG. 6A is an isometric view of a segmented adjustable arm with a locking mechanism, with an internal locking train comprising balls, in accordance with some embodiments of the present invention.

FIG. 6B is a cross sectional view of the segmented adjustable arm with locking mechanism of FIG. 6A.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, the conjunction “or” as used herein is to be understood as inclusive (any or all of the stated options).

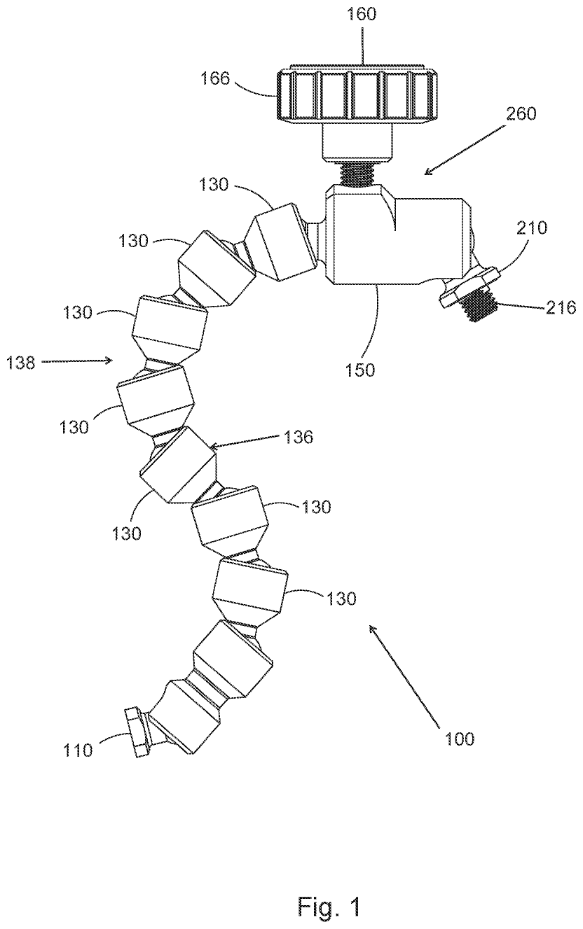

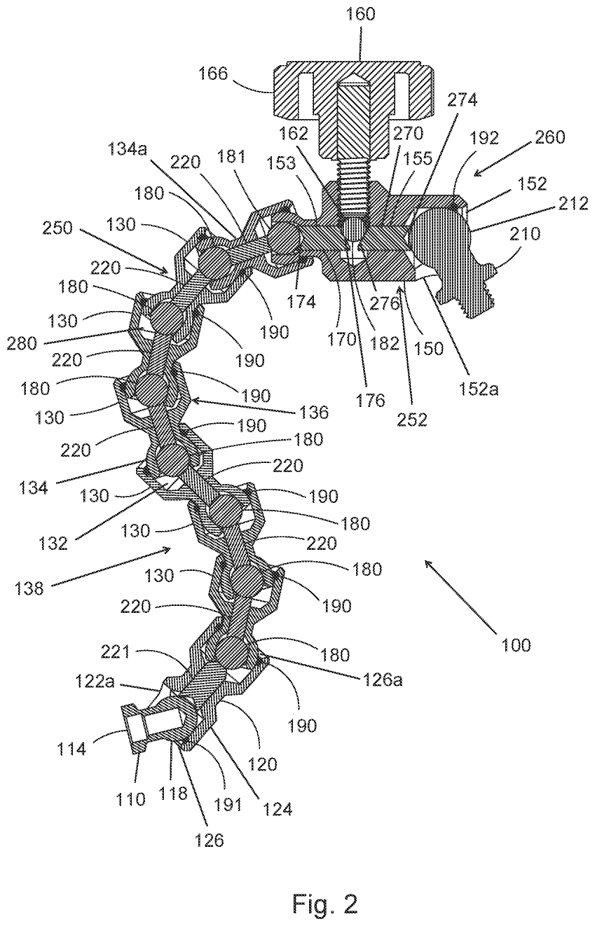

FIG. 1 is a side view of a segmented adjustable arm with a locking mechanism, in accordance with some embodiments of the present invention. FIG. 2 is a cross section view of the segmented adjustable arm with locking mechanism of FIG. 1.

Segmented adjustable arm 100 may include a chain 138 of segments, comprising a plurality of interconnected segments 130, and a locking mechanism 260.

Adjacent ends of each pair of adjacent segments 130 of the segment chain form a ball and socket joint 136, one end of the adjacent ends of each pair of the adjacent segments presenting a spherical or partially spherical plug 134 and the other end of the adjacent ends presenting a socket 132 configured to receive and hold plug 134.

The ball and socket joint may be designed to allow relative motion between the segments of the pair of adjacent segments, e.g., to allow rotation of the plug within the socket 132 while retaining the plug in the socket, thereby facilitating relative motion between the two adjacent segments.

In some embodiments of the present invention, the ball and socket joints 136 may provide arm 100 radial and rotational degrees of freedom. Each pair of adjacent segments may be tilted at an angle relative to each other and/or may be rotated relative to each other. The relative tilt angle and rotation of each pair of adjacent segments of the segment chain 138 may define a desired alignment and orientation of arm 100 in space. For example, arm 100 may be reshaped by tilting one or more of pairs of adjacent segments relative to each other, and/or by rotating one or more of pairs of adjacent segments relative to each other. A maximum tilt between two adjacent segments may be achieved when neck 134a of plug 134 of one of the adjacent segments contacts socket 132 of the other of the adjacent segments. For another example, a maximum tilt between two adjacent segments may be achieved when a distal end of the plug 134 of one of the adjacent segments contacts an internal surface of socket 132 of the other of the adjacent segments.

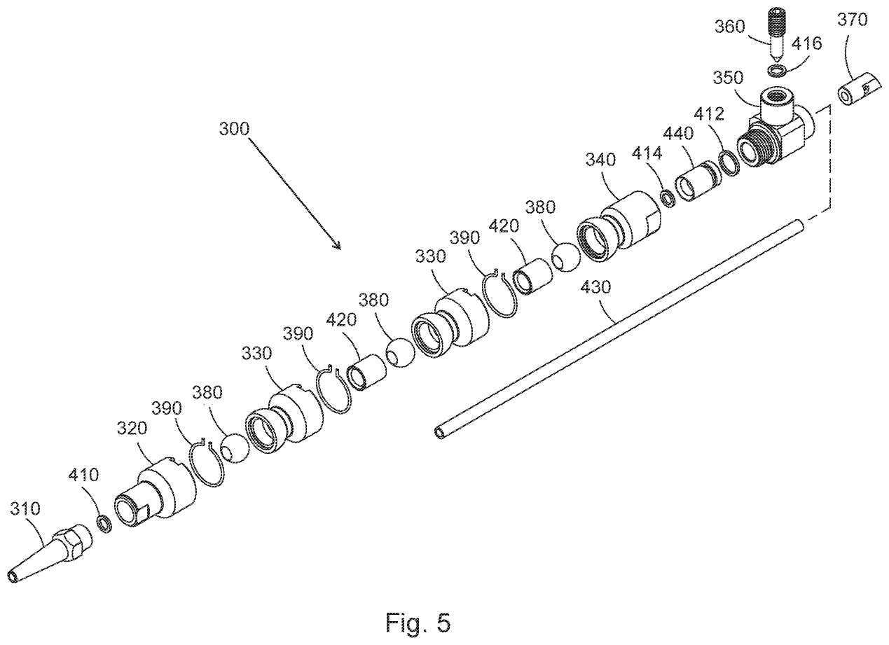

In order to retain the plug within the socket a clip 190 (see also 390 in FIG. 5) may be used (e.g., an annular wire retainer) to prevent inadvertent release of the plug from the socket. In some embodiments of the present invention a holder such as a circular clamp, an elastic ring, a fastener, a hose clamp or any other holder may be used to retain the plug within the socket.

Locking mechanism 260 may include a locking train (not shown in this figure, see 250 in FIG. 2) of one rigid element or a plurality of rigid elements aligned in series along the segment chain, configured, when subjected to applied pressure, to pull the plurality of interconnected segments away from each other, thereby increasing friction at the ball and socket joint between each pair of adjacent segments of the segment chain so as to immobilize motion between the adjacent segments and to fix the segmented adjustable arm in a desired alignment.

A pressure applicator 150 may be provided for applying pressure on the locking train or for releasing the pressure on the locking train, as desired.

Segment chain 138 of arm 100 may be shaped and reshaped in various desired alignments, and a distal end of arm 100 may be oriented, brought to and firmly positioned at a desired point in space. Locking mechanism 260 may fix arm 100 in a desired alignment for maintaining the shape given to arm 100 and the position and orientation of the distal end of arm 100.

In some embodiments of the present invention, arm 100 may hold a load on the distal end and support the load in a desired position in space, when arm 100 is immobilized. Arm 100 may be configured to withstand loads and forces, up to a threshold, while maintaining the arm 100 in its assumed shape. For example, arm 100 may support a mounted camera, a smartphone, a monitor, or another load. Arm 100 may be easily reshaped when pliable, and then immobilized at the desired alignment. For example, when pliable, arm 100 holding a camera may be reshaped to point the camera lens in a desired direction, and then immobilized maintain the camera pointing at the desired direction.

In some embodiments of the present invention, arm 100 may be used in medical procedures. Arm 100 may be shaped according to a desired configuration, aligned in a desired alignment and immobilized to retain the shape and orientation thereof to hold one or more tools in a medical procedure. For example, arm 100 may be used as a utility tool for holding a part of the human body. Arm 100 may be used for holding a surgical tool such as a camera or a clamp.

In some embodiments of the present invention, when arm 100 is pliable, relative motion between each pair of segments of the segment chain is enabled, so as to align arm 100 in a desired alignment. When the pressure applicator is operated the locking mechanism causes arm 100 to be immobilized, keeping arm 100 in a desired alignment.

In some embodiments of the present invention locking mechanism 260 may fix arm 100 by pulling the interconnected segments of segment chain 138 apart, increasing friction and immobilizing relative motion between the segments 130. The locking mechanism may include a locking train (e.g., locking train 250 of FIG. 2) that includes a series of rigid elements (balls 180 and tubular members 220 of FIG. 2 arranged in series along the segmented arm). The locking train may be configured, when subjected to applied pressure (e.g., from locking mechanism 260), to pull the plurality of interconnected segments away from each other. For example, locking train may be configured, when subjected to applied pressure, to push distal segment 110 of the segment chain 138, thereby pulling the interconnected segments away from each other, stretching the segmented arm. In other embodiments of the present invention, arm 100 may comprise a locking train comprising balls such as balls 180. The locking mechanism may fix arm 100 by applying force on the train of balls for immobilizing relative motion between the segments 130.

In some embodiments of the present invention locking mechanism 260 may render arm 100 pliable by relaxing the pressure on the interconnected segments of segment chain 138 apart. The locking train may be configured, when the applied pressure is released or decreased (e.g., locking mechanism 260 releases pressure on the locking train), to reduce the friction at the ball and socket joint 136 between each pair of the adjacent segments of the segment chain 138, so as to enable relative motion between adjacent segments of segment chain 138 and render arm 100 pliable.

in some embodiments of the present application, the locking mechanism 138 may comprise a pressure applicator 150. The pressure applicator 150 may be configured to apply and release pressure on the locking train for fixing arm 100 and rendering arm 100 pliable (respectively). The pressure applicator 150 may comprise a screw 160 that actuates a plunger 170 for applying and releasing pressure on the locking train. Screw 160 may include a knob 166 for enhanced grip of the screw, to apply pressure, when screwed in, on the locking train for fixing arm 100, and to release pressure from the locking train, when screwed out, for rendering arm 100 pliable. In some embodiments of the present invention, screw 160 is oriented substantially orthogonally to the plunger 170 and second plunger 270. For example, pressure applied from screw 160 may be divided substantially equally in amount and at opposite directions between locking train 250 and second locking train 252. In some embodiments ball 176 may be provided juxtaposed to end of the screw 160 for applying substantially equal pressure on both plungers 170 and 270.

In some embodiments of the present invention, the pressure applicator may include a mechanical, electro-mechanical, electromagnetic, pneumatic or hydraulic mechanism, or other mechanism that is configured to apply (and release) pressure,

In some embodiments of the present invention, segment chain 138 may include a distal segment 110 (similar or different in shape to segments 130) located at a distal end of segment chain 138. Distal segment 110 may be configured to be fixed when arm 100 is immobilized, and may be released when arm 100 is relaxed. Distal segment 110 may be designed to form a connector for connecting a load to the adjustable segmented arm 100. Distal segment 110 may comprise a bore (such as bore 114 of FIG. 2) configured to receive an attachment device (e.g., a screw) or an insert of a load, so as to hitch or otherwise firmly, link the load to the distal segment 110 of arm 100. Distal segment 110 may comprise a threaded portion, a magnet/electromagnet a threaded bore or any similar arrangement for connecting a load to the arm.

In some embodiments of the present invention, a segmented adjustable arm may be connected to one or more additional segmented adjustable arms, in parallel or in series. In some embodiments of the present invention, locking mechanism 260 may comprise a second locking train (such as second locking train 252 of FIG. 2). The second locking train may be configured to operate in a direction opposite to a direction of operation of the first locking train. This is so, for example, when the pressure applicator is not positioned at an end of the arm, but at an intermediary position along the arm,

between the proximal end and the distal end of arm 100. The pressure applicator 150 may be thus be designed to apply pressure on the first locking train and on the second locking rain, to immobilize arm 100, and to release the pressure on both locking trains to render arm 100 pliable.

In some embodiments of the present invention, arm 100 may comprise proximal segment 210 at proximal end of arm 100. Proximal segment 210 may he linked to the segment chain in a similar manner as other segments so that it is immobilized when arm 100 is immobilized, and may be moved when arm 100 is pliable. Proximal segment 210 may include an anchoring arrangement (e.g., a threaded portion 216) for anchoring arm 100 to a fixed position (e.g., to a wall, floor, ceiling, a table or other fixed object). Proximal segment 210 may include a magnet, a threaded bore and/or other arrangements for anchoring arm 100.

Segment chain 138 may include interconnected segments such as segment 130, proximal segment 210, distal segment 110 and bi-socketed segment 120, for receiving at both opposite ends a plug. In some embodiments of the present invention, adjustable arm 100 may comprise clips 190, 191 and 192 at the bail and socket joints 136 of the adjustable arm for holding the socket and the plug of the socket and ball joints 136 together, and to prevent socket and ball joint 136 from being disassembled (e.g., when the interconnected segments are pulled apart).

In some embodiment of the present invention, the locking train 250 may comprise one or a plurality of rigid elements arranged in series along the segment chain 138. The rigid elements may comprise a series of balls 180 and tubular members 220, wherein when pressure is applied on the locking train 250, each ball 180 (starting with ball 181 or proximal segment 210) pushes the following tubular member 220, and each tubular member 220 pushes the following ball. When pressure is applied on distal tubular member 221, distal tubular member 221 pushes distal segment 110 so as to pull the plurality of interconnected segments away from each other.

In some embodiments of the present invention, locking train 250 may be inserted within segment chain 138, e.g., within a hollow portion of each interconnected segment of segment chain 138. For example, locking train 250 may extends within a lumen 280 defined within the segment chain 138. Ball 180 may be positioned within plug 134, and tubular members 220 may be positioned between the balls 180.

Tubular member 220 may be configured to optimize contact with ball 180 for optimal transfer of pressure to ball 180. For example, tubular member 220 may have a concave surface at each of its opposite ends for maximizing contact surface with ball 180. Tubular member 220 may be provided in a form of a cylinder or have an elongated body with a circular, oval polygonal, or other cross section.

In some embodiments of the present invention, proximal segment 210 may rotate and tilt (radially) in relation to pressure applicator 150. Pressure applicator 150 may have socket 152 configured to receive and hold ball shaped end 212 of proximal segment 210. In some embodiments of the present invention, proximal segment 210 may be confined to tilt along an axis defined by groove 152a of pressure applicator socket 152,

In some embodiments of the present invention, Distal segment 110 may rotate and tilt (radially) in relation to bi-socketed segment 120. Bi-socketed segment 120 may have distal socket 126 configured to receive and hold ball shaped end 118 of distal segment 110. Bi-socketed segment 120 may have proximal socket 126a configured to receive and hold plug 134 of segment 130. In some embodiments of the present invention, distal segment 110 may be confined to tilt along an axis defined by groove 122a of bi-socketed segment 120.

In some embodiments of the present invention, the adjustable arm and/or parts of the adjustable arm such as the one or a plurality of interconnected segments, the locking mechanism, the one or a plurality of rigid elements such as the balls and tubular members, and any other part or parts of the adjustable arm may comprise materials such as metal, metal composite, plastic, polymers, carbon fiber, composite materials and any other suitable material.

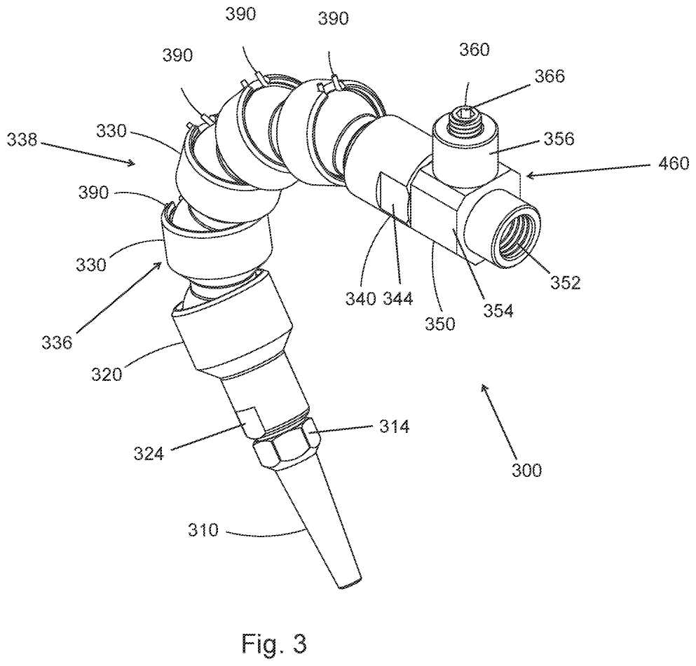

FIG. 3 is an isometric view of a segmented adjustable arm with a locking mechanism, with an internal conduit for delivering and dispensing fluid, in accordance with some embodiments of the present invention. In some embodiments of the present invention, the internal conduit may be used as a passage for cables such as electrical wires and optical cables. In some embodiments of the present invention, the internal conduit may be used for delivering pressurized air and other fluids. In other embodiments of the present invention, the internal conduit may not be used for delivering fluid (e.g., the hollow structure may have mechanical properties for supporting arm 300, and not specifically designed for delivering fluid).

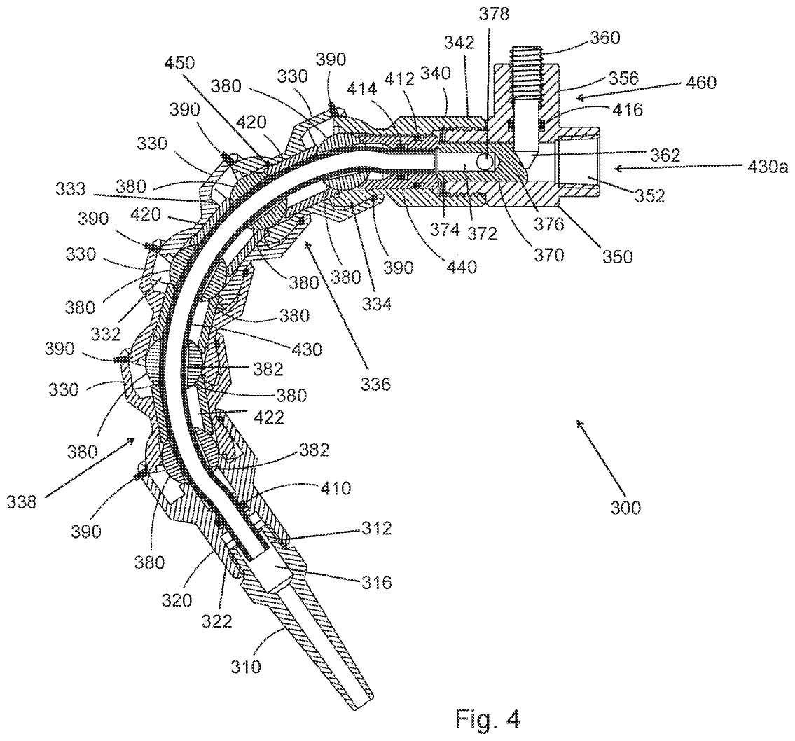

FIG. 4 is cross section view of the segmented adjustable arm with locking mechanism of FIG. 3, and FIG. 5 is an exploded view of the segmented adjustable arm with locking mechanism of FIG. 3.

Segmented adjustable arm 300 may include a segment chain 338, comprising a plurality of interconnected segments 330, and a locking mechanism 460. Segmented chain 338 of arm 300 may be shaped and aligned according to a user's desire, and a distal end of arm 300 may be oriented and positioned at a desired point in space. Locking mechanism 460 may fix arm 300 in a desired alignment for keeping the shape given to arm 300 and the orientation of the distal end of arm 300.

In some embodiments of the present invention, arm 300 may be easily aligned when pliable, and then immobilized at the desired alignment.

In some embodiments of the present invention, a conduit (e.g., a tube) may be provided, extending within arm 300 (see FIG. 4-FIG. 5) for dispensing fluid. An outlet of the conduit, e.g., nozzle 310 may be located at a distal portion of arm 300, so that when the distal end of arm 300 is pointed at a target (e.g., a workpiece to be machined), fluid may be dispensed on the target. Fluid may enter arm 300 through inlet 352 of pressure applicator 350 to he dispensed at nozzle 310.

Similarly to the adjustable arm of FIG. 1, according to some embodiments of the present invention, adjustable arm 300 may comprise a segment chain 338. Adjacent ends of each pair of adjacent segments of the segment chain 338 may form a ball and socket joint 336, allowing relative motion (e.g., tilt, rotation) between the segments of that pair. The ball and socket joints 336 may provide adjustable arm 300 with radial and rotational degrees of freedom. Each pair of adjacent segments may be tilted at an angle relative to each other, and each pair of adjacent segments may be rotated relative to each other. The relative tilt angle and rotation of each pair of adjacent segments of the segment chain 338 may define the alignment and orientation of arm 300 in space. According to some embodiments of the present invention locking mechanism 460 may be configured to immobilize arm 300 by pulling the interconnected segments of segment chain 338 apart, to increase friction between the jointed segments. The locking mechanism may include a locking train 450 that includes a series of rigid elements (e.g., balls 380 and tubular members 420). The locking train 450 may be configured, when subjected to applied pressure (e.g., from locking mechanism 460), to push a distant segment at a distal end of the arm, thereby pulling the interconnected segments away from each other. increasing friction at the bail and socket joint 336 between each pair of the adjacent segments (by pulling the segments away from each other), so as to immobilize relative motion between the adjacent segments and to immobilize the segment chain 338 fixing adjustable arm 300 in a desired alignment.

In some embodiments of the present invention locking mechanism 460 may render adjustable arm 300 pliable by relaxing the pressure of the pressure applicator 350 thereby reducing the friction between the interconnected segments of segment chain 338.

In some embodiments of the present application, the locking mechanism 460 may include a pressure applicator 350. The pressure applicator 350 may be configured to apply or release pressure on the locking train for fixing adjustable arm 300 or for rendering arm 300 pliable (respectively). The pressure applicator 350 may include screw 360 that actuates a plunger inside the applicator (370 in FIG. 4) for applying and releasing pressure on the locking train. Screw 360 (e.g., Allen screw, with hexagonal socket 366, operable by a hex key) may be screwed in to apply pressure on the locking train for immobilizing adjustable arm 300, and screwed out, to release pressure from the locking train for relaxing adjustable arm 300.

In some embodiments of the present invention, segment chain 338 may include a distal segment 320 at a distal end of segment chain 338. Distal segment 320 may be fixed when arm 300 is immobilized, and may move when arm 300 is relaxed so as to orient nozzle 310 (e.g. in the direction of a workpiece).

In some embodiments of the present invention, segment chain 338 may include a proximal segment 340 at a proximal end of segment chain 338. Proximal segment 340 may be linked to pressure applicator 350. Proximal segment 340 may include a spherical or partially spherical plug 334. A socket 332 of proximal segment 340 may receive and hold the plug of proximal segment 340.

In some embodiments of the present invention, adjustable arm 300 may comprise clips 390 at the ball and socket joints 336 of arm 300 for holding the socket and ball joints 336 together, and to prevent socket and ball joint 336 from being disassembled (e.g., when the interconnected segments are pulled apart).

In some embodiments of the present invention, the locking train 450 may include a bore that extends within the locking train 450, defining an internal passage 430a. The internal passage 430a may be used for transferring fluid through arm 300. Each ball 380 and tubular member 420 of the locking train 450 may comprise a bore, so that these bores are linked in series, defining an elongated internal passage 430a through adjustable arm 300. Plunger 370 too may be hollow and include one or a plurality of side holes 378 fluidically linked to internal passage 430a. Fluid may he introduced into internal passage 430a pass through inlet 352, through plunger 370, along internal passage 430a within the locking train 450, and be dispensed from nozzle 310.

In some embodiments of the present invention, tube 430 may be provided within the internal passage 430a for passing fluid. The tube 430 may include an inlet at a proximal portion of arm 300 (e.g., inlet 352), and an outlet at a distal portion of the adjustable arm (e.g., nozzle 310). Tube 430 may be made of an elastic material that may bend elastically following a curvature of arm 300 and that can withstand high pressures (e.g., 70 Bar) and high temperatures (e.g., 70 degrees Celsius).

In some embodiments of the present invention, adjustable arm 300 may comprise gaskets, (e.g., O-rings) and sealants to prevent fluid leakage. Distal segment 320 of adjustable arm 300 may have a groove configured to receive O-ring 410. O-ring 410 of distal segment 320 may connect tube 430. Pressure applicator 350 may have a groove configured to receive O-ring 416. O-ring 416 of locking segment 350 may connect threaded insert 460. First tubular member 440 may have a groove configured to receive O-ring 414, that connects tube 430. First segment 340 may have a groove configured to receive O-ring 412, that connects first tubular member 440.

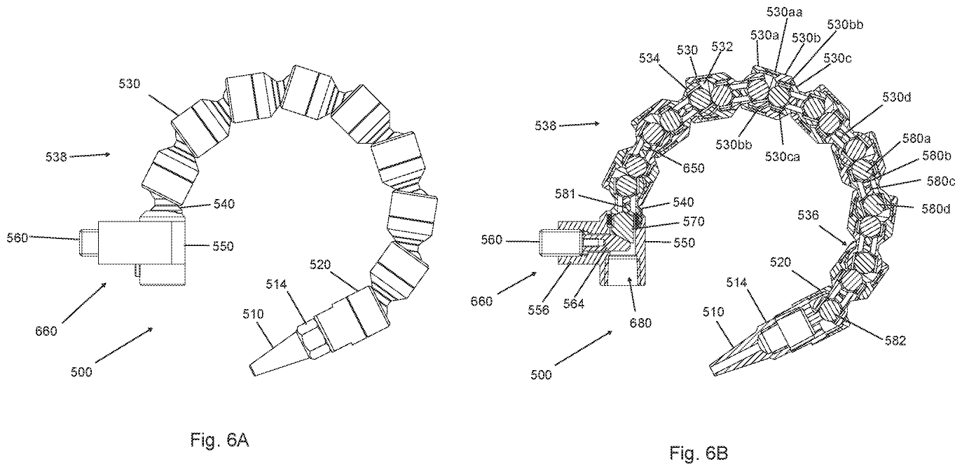

FIG. 6A is an isometric view of a segmented adjustable arm with a locking mechanism, with an internal locking train comprising balls, in accordance with some embodiments of the present invention. FIG. 6B is a cross sectional view of the segmented adjustable arm with locking mechanism of FIG. 6A.

Segmented adjustable arm 500 may include a segment chain 538, comprising a plurality of interconnected segments 530, and a locking mechanism 660. Segmented chain 538 of arm 500 may be shaped and aligned according to a user's desire, and a distal end of arm 500 may be oriented and positioned at a desired point in space. Locking mechanism 660 may fix arm 500 in a desired alignment for keeping the shape given to arm 500 and the orientation of the distal end of arm 500.

According to some embodiments of the present invention, adjustable arm 500 may comprise a segment chain 538. Adjacent ends of each pair of adjacent segments of the segment chain 538 may form a ball and socket joint 536, allowing relative motion (e.g., tilt, rotation) between the segments of that pair. The ball and socket joints 536 may provide adjustable arm 500 with radial and rotational degrees of freedom. Each pair of adjacent segments may be tilted at an angle relative to each other, and each pair of adjacent segments may be rotated relative to each other. The relative tilt angle and rotation of each pair of adjacent segments of the segment chain 538 may define the alignment and orientation of arm 500 in space. According to some embodiments of the present invention locking mechanism 660 may be configured to immobilize arm 500 by pulling the interconnected segments of segment chain 538 apart, to increase friction between the jointed segments. The locking mechanism may include a locking train 650 that includes a series of rigid elements (e.g., balls 580a, 580b, 580c, 580d arranged in series). The locking train 650 may be configured, when subjected to applied pressure (e.g., from locking mechanism 660), to push a distant segment at a distal end of the arm, thereby pulling the interconnected segments away from each other, increasing friction at the ball and socket joint 536 between each pair of the adjacent segments (by pulling the segments away from each other), so as to immobilize relative motion between the adjacent segments and to immobilize the segment chain 538 fixing adjustable arm 500 in a desired alignment.

In some embodiments of the present invention locking mechanism 660 may render adjustable arm 500 pliable by relaxing the pressure of the pressure applicator 550 thereby reducing the friction between the interconnected segments of segment chain 538.

In some embodiments of the present application, the locking mechanism 660 may include a pressure applicator 550. The pressure applicator 550 may be configured to apply or release pressure on the locking train for fixing adjustable arm 500 or for rendering arm 500 pliable (respectively). The pressure applicator 550 may include screw 560 that actuates the locking train plunger 570 for applying and releasing pressure on the locking train. Screw 560 may be screwed in, driving screw plunger 564 into pressure applicator 550. When pushed into pressure applicator 550, screw plunger 564 may apply pressure on locking train plunger 570, driving locking train plunger 570 towards proximal ball 581 to apply pressure on the locking train. When pressure is applied on the locking train, adjustable arm 500 is immobilized. Screw 560 is screwed out, to release pressure from the locking train for relaxing adjustable arm 500.

In some embodiments of the present invention, segment 538 may include a distal segment 520 at a distal end of segment chain 538. Distal segment 520 may he fixed when arm 500 is immobilized, and may move when arm 500 is relaxed so as to orient nozzle 510 (e.g. in the direction of a workpiece). Nozzle 510 may be screwed to distal segment 520. Different parts may be used at the distal end of arm 500 instead of Nozzle 510, for example, a fixture may be used to hold an attachment such as a camera.

In some embodiments of the present invention, segment chain 538 may include a proximal segment 540 at a proximal end of segment chain 538. Proximal segment 540 may be linked to pressure applicator 550. Proximal segment 540 may include a spherical or partially spherical plug 534. A socket 532 of proximal segment 540 may receive and hold the plug of proximal segment 540.

In some embodiments of the present invention, segment 530 may comprise multiple subsegments 530a-c. Proximal subsegment 530a may comprise socket 532 and ball 580a. Proximal subsegment 530a may comprise proximal segment threaded portion 530aa for connecting with middle subsegment 530b at middle segment proximal threaded portion 530ba. Middle subsegment 530h may comprise tubular lumen 530d including balls 580h and 580c. Middle subsegment 530h may comprise middle subsegment distal threaded portion 530bb connecting with distal subsegment 530c a distal subsegment threaded portion 530ca. Distal subsegment 530c may comprise plug 534 and ball 580d. Segment chain 538 may be assembled by screwing the subsegments (such as subsegments 530a-c) together and inserting the balls (such as balls 580a-d) of the locking train.

In some embodiment of the present invention, the locking train 650 may comprise one or a plurality of rigid elements arranged in series along the segment chain 538. The rigid elements may comprise a series of balls (such as balls 580a-d), wherein when pressure is applied on the locking train 650, each ball pushes the following ball. When pressure is applied on distal ball 582, distal ball 582 pushes distal segment 520 so as to pull the plurality of interconnected segments of segment chain 538 away from each other and fix arm 500.

In some embodiments of the present invention, locking train 650 may be inserted within segment chain 538, e.g., within a hollow portion of each interconnected segment of segment chain 538. For example, locking train 650 may extends within a lumen 680 defined within the segment chain 538. Ball 580d may be positioned within plug 534, Ball 580a may be positioned within socket 532, and ball 580b and ball 580c may he positioned in tubular lumen 530d of segment 530. Ball 580a and bail 580d may be bigger than ball 580b and ball 580c.

In some embodiments of the present invention some of the balls may have different sizes. In other embodiments of the present invention, the balls may have substantially the same size.

Different embodiments are disclosed herein. Features of certain embodiments may he combined with features of other embodiments. Thus, certain embodiments may be combinations of features of multiple embodiments. The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A segmented adjustable arm device comprising:
a segment chain comprising a plurality of interconnected segments, wherein adjacent ends of each pair of adjacent segments of the segment chain form a ball and socket joint, one of the adjacent ends of each pair of the adjacent segments presenting a spherical or partially spherical plug and the other end of the adjacent ends presenting a socket configured to receive and hold the plug, allowing relative radial motion between the segments of that pair; and
a locking mechanism comprising:
a locking train that includes one or a plurality of rigid elements arranged in series extending along the segment chain, configured, when subjected to applied pressure, to pull the plurality of interconnected segments away from each other, and increase friction at the ball and socket joint between each pair of the adjacent segments so as to immobilize motion between adjacent segments of the segment chain to fix the segmented adjustable arm in a desired alignment, and configured, when the pressure is released, to reduce the friction at the ball and socket joint between each pair of the adjacent segments of the segment chain, to render the segmented adjustable arm pliable; and
a pressure applicator to apply or release the pressure.

2. The device of claim 1, wherein the locking train is configured, when subjected to applied pressure, to push a distal segment of the segment chain.

3. The device of claim 1, wherein the locking train is inserted in the segment chain.

4. The device of claim 3, wherein the locking train extends within a lumen defined within the segment chain.

5. The device of claim 3, wherein the plurality of rigid elements comprises balls arranged in series.

6. The device of claim 5 wherein the plurality of rigid elements comprises balls and tubular members arranged in series.

7. The device of claim 1, wherein a bore extends within the locking train, defining an internal passage.

8. The device of claim 7, wherein a tube extending within the internal passage.

9. The device of claim 8, wherein the tube comprises an inlet at a proximal portion of the adjustable arm, and an outlet at a distal portion of the adjustable arm.

10. The device of claim 9, wherein the outlet is located at a distal end of the adjustable arm.

11. The device of claim 1, wherein the pressure applicator comprises a plunger for applying the pressure on one of the rigid elements of the locking train.

12. The device of claim 11, wherein the pressure applicator comprises a screw configured to actuate the plunger.

13. The device of claim 12, wherein the screw is oriented substantially orthogonally to the plunger.

14. The device of claim 11, wherein the plunger is hollow, forming part of the internal passage.

15. The device of claim 11, further comprising a second locking train.

16. The device of claim 15, wherein the second locking train is configured to operate in a direction opposite to a direction of operation of the locking train.

17. The device of claim 1, wherein at east one segment of the segment chain comprises a plurality of subsegments.

18. The device of claim 17 wherein the subsegments comprise threaded portions.
